(19) United States
(12) Patent Application Publication
(10) Pub. No.: US 2022/0113294 A1
(43) Pub. Date: Apr. 14, 2022

(54) Pathogen Detection Apparatus and Method

(71) Applicant: Yonatan Gerlitz, Herzliya (IL)

(72) Inventor: Yonatan Gerlitz, Herzliya (IL)

(21) Appl. No.: 17/498,441

(22) Filed: Oct. 11, 2021

Related U.S. Application Data
(60) Provisional application No. 63/090,012, filed on Oct. 9, 2020.

(51) Int. Cl. G01N 33/497; G01N 27/49

(52) U.S. Cl. CPC G01N 33/497; G01N 2800/26; G01N 27/49

(57) ABSTRACT

Pathogen detection includes providing a sensor having first conductive lines alternated with second conductive lines, the first and second lines being formed over a substrate providing electrical isolation between the first and second lines and being at least partially exposed. Via voltage circuitry, a voltage difference is applied between the first and second lines. A user's breath is applied to the sensor and contacts exposed parts of the first lines simultaneous to exposed parts of the second lines. Pathogens from the user's breath bridge the electrical isolation between an individual first line and an opposing, individual second line and cause a short circuit. Via a comparator or controller, a current is detected flowing in the first and second lines due to the short circuit through the pathogens. A warning module indicates that the comparator or controller detected that the current is above a warning threshold.

### CROSS REFERENCE TO RELATED APPLICATIONS

This application claims the benefit under 35 U.S.C. § 119 to U.S. Provisional Pat. App. No. 63/090,012, filed on Oct. 9, 2020 and entitled “Pathogen Detection Apparatus and Method”, which is incorporated herein by reference.

### BACKGROUND

The COVID-19 and other world-wide epidemics are raising an urgent need for a low-cost, disposable device for screening a large scale of population in order to identify and isolate infected persons.

### SUMMARY

A pathogen detection method includes providing a sensor having first conductive lines alternated with second conductive lines, the first and second lines being formed over a substrate providing electrical isolation between the first and second lines and being at least partially exposed. Via voltage circuitry, a voltage difference is applied between the first and second lines. The method includes applying a user's breath to the sensor and contacting exposed parts of the first lines simultaneous to exposed parts of the second lines. Pathogens from the user's breath bridge the electrical isolation between an individual first line and an opposing, individual second line and cause a short circuit. Via a comparator or controller, a current is detected flowing in the first and second lines due to the short circuit through the pathogens. The method includes, via a warning module, indicating that the comparator or controller detected the current is above a warning threshold.

A pathogen detection apparatus includes a sensor having first conductive lines alternated with second conductive lines, the first and second lines being formed over a substrate providing electrical isolation between the first and second lines and being at least partially exposed such that, during use, a user's breath contacts exposed parts of the first lines simultaneous to exposed parts of the second lines. Voltage circuitry is configured to apply, during use, a voltage difference between the first and second lines. A comparator or controller is configured to detect when a current flows in the first and second lines due to a short circuit through pathogens from the user's breath bridging the electrical isolation between an individual first line and an opposing, individual second line. A warning module is configured to indicate when the comparator or controller detects the current flow above a warning threshold.

The features, functions, and advantages that have been discussed can be achieved independently in various implementations or may be combined in yet other implementations further details of which can be seen with reference to the following description and drawings.

### BRIEF DESCRIPTION OF THE DRAWINGS

Some implementations are described below with reference to the following accompanying drawings.

FIG. 1 is a top view of a schematic illustration of one example of a sensor built from a substrate, such as a silicon chip, with alternated voltage lines for capturing viruses and/or microbes.

FIG. 2 is a perspective, exploded view of a schematic illustration of one example of a modification of the sensor in FIG. 1 to include two layers in a net for capturing viruses and/or microbes.

FIG. 3 is a perspective, exploded view of a schematic illustration of the net of FIG. 2, with through-holes between the net lines, to be used as a filter.

FIG. 4 is a perspective view of a schematic illustration of one example of an apparatus with a sensor, such as the sensor of FIG. 1 or 2, installed inside.

FIG. 5 is a block diagram of one example of a low cost electronic circuit associated with a sensor, such as the sensors of FIGS. 1-3.

FIG. 6 is a block diagram of one example of a more sophisticated electronic circuit for a quantitative measurement associated with a sensor, such as the sensors of FIGS. 1-3.

### DETAILED DESCRIPTION

Numerous circumstances arise in which a desire exists to screen people quickly who are potentially infected with a pathogen. During the COVID-19 pandemic, screening procedures often included measuring body temperature and/or completing a survey about health conditions. However, such screening cannot detect infected persons before the onset of illness symptoms. Large numbers of people pass through transportation centers, employment centers, health care facilities, etc. where infected persons could potentially transmit pathogens to numerous other people before illness symptoms begin. Accordingly, these circumstances raise an urgent need for a device to screen large populations in an economical manner in order to identify and isolate infected persons. Once screening identifies an infected person, known higher cost and more time-consuming testing may determine the nature of the illness.

Some examples described herein operate on the principle that infected persons' exhaled breath may carry pathogens that transmit an illness to other persons before other symptoms begin. But, exhaled pathogens might also be detected with a suitable device. Some examples herein provide a pathogen detection apparatus with a sensor including first conductive lines alternated with second conductive lines such that, during use, a user's breath contacts exposed parts of the first lines simultaneous to exposed parts of the second lines. A voltage difference is applied between the first and second lines. A comparator or controller may detect when a current flows in the first and second lines due to a short circuit through pathogens from the user's breath bridging electrical isolation between an individual first line and an opposing, individual second line. A warning module may indicate when the comparator or controller detects the current flow above a warning threshold. In this manner, infected persons may be identified almost instantly simply by collecting exhaled breath.

Therefore, instead of relying on samples of bodily fluid to conduct costly testing that gives time-delayed results, some examples herein yield very quick results using low-cost devices. In one implementation, various methods and apparatuses described herein use a disposable electronic chip and device for fast screening of a population suffering infection by a pathogen.

According to one of the apparatuses described herein, an electronic chip is capable of detecting an abnormal quantity of pathogens, such as viruses and/or microbes, in the breath. A device containing this chip is used to send an alert once an abnormal quantity of pathogens, such as viruses and/or microbes, is detected. While viruses and/or microbes are chiefly discussed herein, other categories of pathogens may be detected.

According to another one of the apparatuses described herein, an electronic chip is built on a substrate, such as a silicon wafer, and constructed of alternated conductive lines with isolation between the lines. Voltage is applied to the chip to create a voltage difference of 3 or more volts between opposing lines.

As an example, all the higher voltage lines may be formed from a continuous conductive material. All the lower voltage lines may be formed from the same or a different continuous conductive material separated from the higher voltage lines. The lines' output may be connected to an amplifier that can be part of the chip or can be a separate chip. The output of the amplifier may be connected to a comparator, pre-set to healthy peoples' pathogen level in the breath. The comparator issues an alarm once the level of the pathogens are above the pre-set level.

The electronic chip may be protected by a removable protective layer which can be made, for example, of a flexible thin plastic material. Once the protective layer is removed and a person blows on the chip, pathogens may create a short circuit between the alternated conductive lines and produce a current. The current created is proportional to the number of pathogens in the breath and is used to identify an infected person and to issue an alarm.

The distance between the conductive lines may be 50 nanometers (nm) or less, so the smallest virus may create a short circuit between the lines, while for molecules, such as H₂O, which are much smaller, no short circuit will be created.

In the case that the device is intended to identify only viruses, a filter that allows only particles less than 500 nm in size may be placed in front of the sensitive area of the sensor. This arrangement might also dramatically reduce false positive detection of an infection.

A modified chip may be built of two layers of orthogonal lines, with isolation between the layers, including at the crossing of the lines. Holes formed, such as by etching, through the isolation between the lines of the upper layer may expose the orthogonal lines of the second layer, thus creating a net of voltage lines that may more effectively detect pathogens.

Another modification may provide through-holes between the net lines of the modified chip, so that air can pass through. By assembling a matrix of several chips arranged side-by-side, a filter may be fashioned that will not allow pathogens to pass through.

A pathogen detection device may include an ATD (analog-to-digital) converter or a VTF (voltage-to-frequency) converter and micro-processor and a display to measure and display quantitative measurement of the number of pathogens in addition to issuing an alarm when above a threshold, as described above.

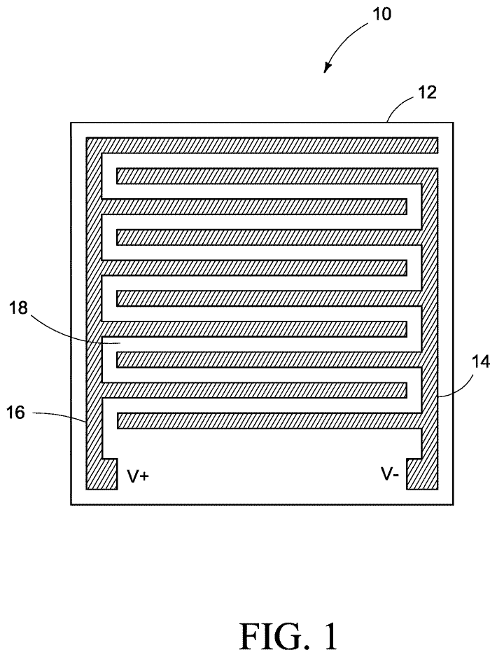

FIG. 1 shows an example sensor 10 built from a substrate 12, such as a silicon chip, with alternated voltage lines 16 and 14 formed over substrate 12. During use, lines 16 have a higher voltage and lines 14 have a lower voltage, creating a voltage difference. Substrate 12 provides isolation 18 between the lines.

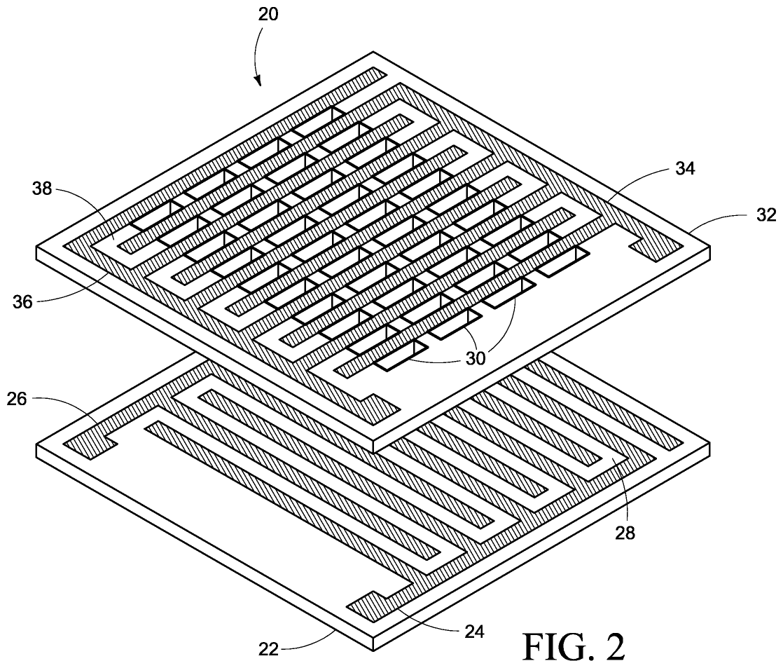

FIG. 2 shows an example sensor 20 modified to include a net of two layers of alternated voltage lines (24/26 and 34/36) and isolation layer 32 in between layers. Voltage lines 24/26 and 34/36 are arranged in the same manner as voltage lines 14/16 in FIG. 1, but voltage lines 24/26 are oriented in a direction orthogonal to voltage lines 34/36. Isolation layer 32 has holes 30 to expose portions of voltage lines 24/26 on substrate 22. Each of the two layers of alternated voltage lines (24/26 and 34/36) have isolation (28 and 38) between the lines.

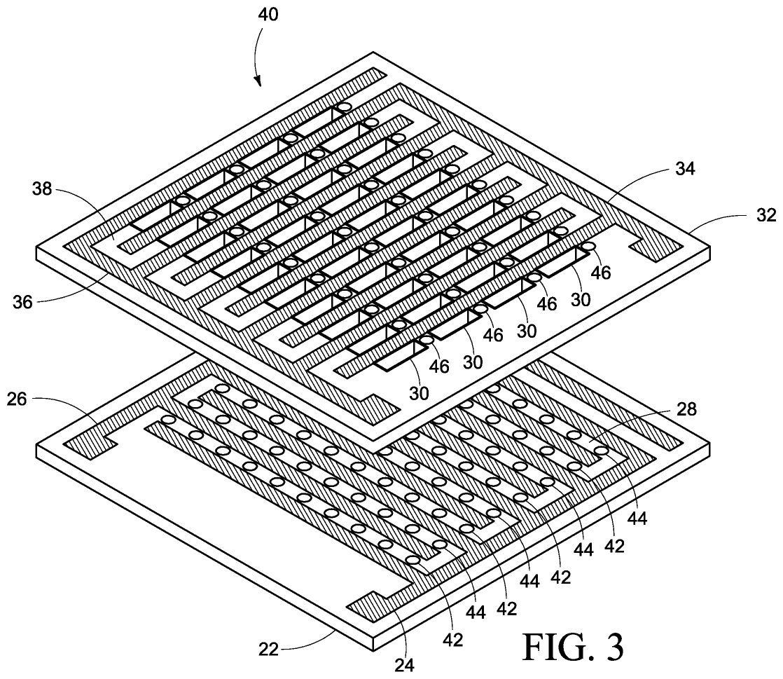

FIG. 3 shows an example sensor 40 with the net of two layers of voltage lines from FIG. 2 modified to include through-holes 42, 44, and 46 between the net lines to be used as a filter. Sensor 40 has the same substrate 22, two layers of alternated voltage lines 24/26 and 34/36, and isolation layer 32 as in sensor 20 in FIG. 2.

While holes 30 through isolation layer 32 expose portions of voltage lines 24/26 on substrate 22, holes 30 also expose holes 42 that are formed through substrate 22. Thus, holes 30 aligned with holes 42 allow air passage through sensor 40 to be used as a filter. Also, holes 46 through isolation layer 32 align with holes 44 through substrate 22 to allow air passage through sensor 40 to be used as a filter. As an example, holes 42, 44, 46 may have a width of 40 nm or less to allow air, water, and other molecules through, but not most pathogens. Holes 30 may have a width of 50 nm or less and a length of 150 nm or less. Several sensors 40 may be placed side-by-side in a matrix and provide sufficient surface area for breathing. Exhaled pathogens may be destroyed when they short circuit between voltage lines 34/36 or 24/26 or, otherwise, will not pass through holes 42, 44, 46.

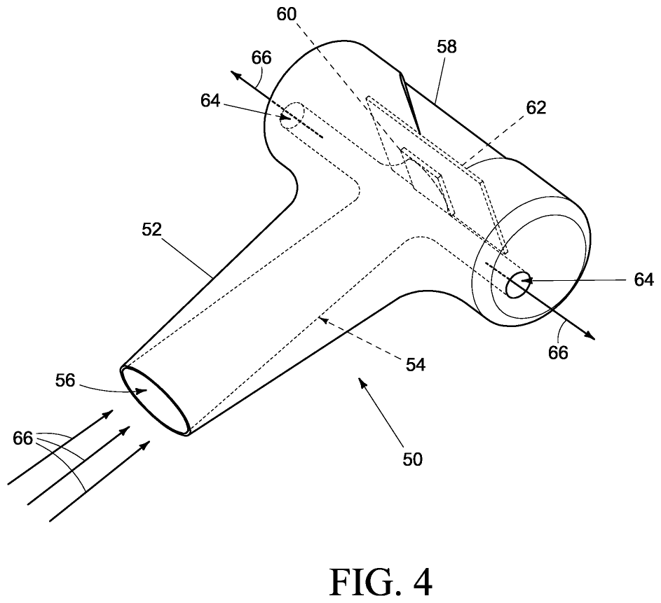

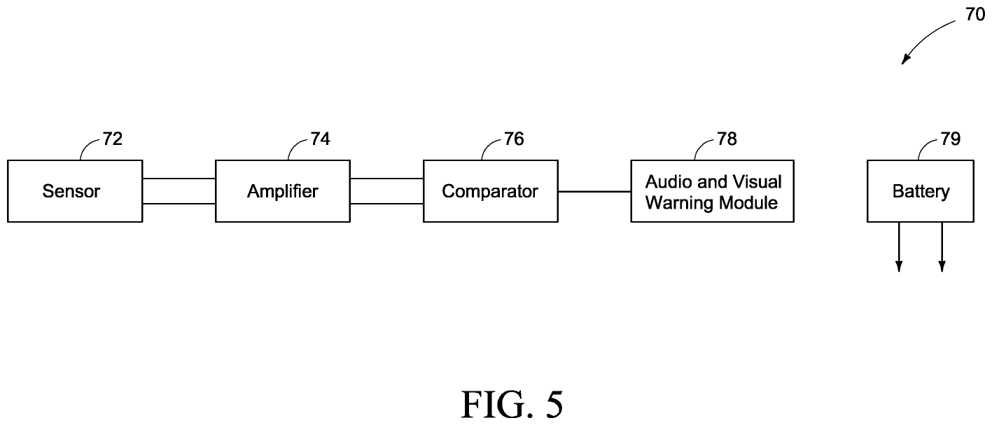

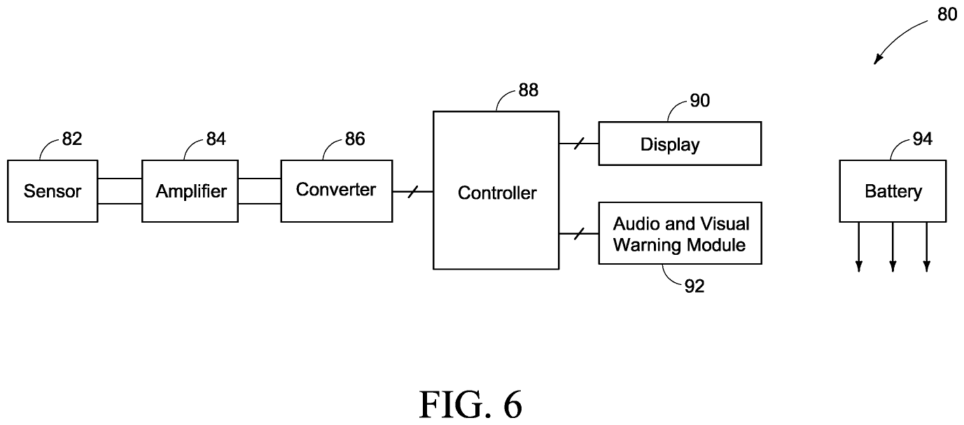

FIG. 4 shows an apparatus 50 with sensor 10 or 20 of FIG. 1 or 2 installed inside as a sensor 60. Other sensors may instead be installed. Apparatus 50 has a housing 52, such as made from plastic, with an inlet 56 to be placed in the patient's mouth for blowing air inside. Interior cone 54 directs the air flow 66 to sensor 60 and out through outlets 64. Sensor 60 is installed on an electronic board 62, such as a printed circuit board, which may include one of the circuits described in FIGS. 5 and 6.

In the event that electronic board 62 includes the circuit of FIG. 6 with quantitative measurement, apparatus 50 includes a display 58, such as an LCD display, to show the measurement. In the event that electronic board 62 includes the circuit of FIG. 5, apparatus 50 might include only a LED light and/or a speaker to provide an alarm. Sensor 60 may have a protective thin flexible plastic layer placed thereon to be pulled out before use (not shown).

FIG. 5 shows an example apparatus 70 that includes a low cost electronic circuit without quantitative measurement. A sensor 72 (such as sensors 10, 20, or 40 described in FIGS. 1-3) is connected to an amplifier 74. Amplifier 74 output is connected to a comparator 76 with a selected level of comparison to activate a warning module 78 when the signal is higher than the selected level. The circuit may be powered by a battery 79, such as a small lithium battery. Though apparatus 70 includes amplifier 74, an amplifier might be left out of other apparatuses. Similarly, though warning module 78 provides both an audio and a visual indication, a warning module might provide only audio or only visual indication in other apparatuses. Further, though apparatus 70 includes battery 79, an alternate power source might be used to energize voltage circuitry in a known manner and apply a voltage difference between voltage lines of sensor 72.

FIG. 6 shows an example apparatus 80 that includes an electronic circuit for quantitative measurement. A sensor 82 (such as sensors 10, 20, or 40 described in FIGS. 1-3) is connected to an amplifier 84. The output of amplifier 84 is connected to a converter 86, such as an ATD or VTF converter, which is connected to a controller 88, such as a microprocessor. The measurement of the current by controller 88 is displayed on a display 90, such as an LCD. In case of high levels of current—above a selected comparison level—controller 88 will activate a warning module 92 to make an alarm. As shown in FIG. 6, converter 86, display 90, and warning module 92 may be connected to controller 88 via a bus. Other forms of connection are conceivable. The circuit may be powered by a battery 94, such as a small lithium battery. Though apparatus 80 includes amplifier 84, an amplifier might be left out of other apparatuses. Similarly, though warning module 92 provides both an audio and a visual indication, a warning module might provide only audio or only visual indication in other apparatuses. Further, though apparatus 80 includes battery 94, an alternate power source might be used to energize voltage circuitry in a known manner and apply a voltage difference between voltage lines of sensor 82.

Apparatuses and Methods

The discoveries described herein identify a number of solutions that may be implemented in apparatuses and methods also described herein. Multiple solutions may be combined for implementation, enabling still further apparatuses and methods. The inventors expressly contemplate that the various options described herein for individual apparatuses and methods are not intended to be so limited except where incompatible with other apparatuses and methods. The features and benefits of individual apparatuses herein may also be used in combination with methods and other apparatuses described herein even though not specifically indicated elsewhere. Similarly, the features and benefits of individual methods herein may also be used in combination with apparatuses and other methods described herein even though not specifically indicated elsewhere.

Method A includes providing a sensor including first conductive lines alternated with second conductive lines, the first and second lines being formed over a substrate providing electrical isolation between the first and second lines and being at least partially exposed. Via voltage circuitry, a voltage difference is applied between the first and second lines. The method includes applying a user's breath to the sensor and contacting exposed parts of the first lines simultaneous to exposed parts of the second lines. Pathogens from the user's breath bridge the electrical isolation between an individual first line and an opposing, individual second line and causing a short circuit. Via a comparator or controller, a current is detected flowing in the first and second lines due to the short circuit through the pathogens. The method includes, via a warning module, indicating that the comparator or controller detected the current is above a warning threshold.

Additional features may be implemented in Method A. By way of example, the exposed parts of the first and second lines may form a sensitive area of the sensor. Within the sensitive area, the first lines may be separated from the second lines by a distance of 50 nanometers or less.

The sensor may include third conductive lines alternated with fourth conductive lines. The third and fourth lines may be formed over an isolation layer providing electrical isolation between the third and fourth lines and be at least partially exposed.

The third and fourth lines may be formed at elevational levels over the first and second lines. The isolation layer may provide electrical isolation between the third/fourth lines and the first/second lines.

The voltage circuitry may also apply the voltage difference between the third and fourth lines. Accordingly, the user's breath to the sensor may also contact exposed parts of the third lines simultaneous to exposed parts of the fourth lines. The pathogens from the user's breath may also bridge the electrical isolation between an individual third line and an opposing, individual fourth line and cause another short circuit. Therefore, the comparator or controller may also detect a current flowing in the third and fourth lines due to the other short circuit through the pathogens.

Holes may be formed through the isolation layer between the third and fourth lines and provide the exposed parts of the first and second lines.

Holes may be formed through the isolation layer between the third and fourth lines, holes may be formed through the substrate between the first and second lines, and the isolation layer holes may be aligned with the substrate holes. As a result, Method A may include applying the user's breath through the aligned holes with the sensor acting as a pathogen filter.

Method A may further include receiving the user's breath at an inlet of a conduit and directing the user's breath through a channel of the conduit to the sensor.

Method A may further include electrically connecting at least one electrical power source to the voltage circuitry.

Method A may further include, via a filter, blocking particles having a size of 500 nanometers or greater from reaching the sensor.

The described additional features of Method A may also be implemented in other apparatuses and methods herein.

Apparatus B includes a sensor having first conductive lines alternated with second conductive lines, the first and second lines being formed over a substrate providing electrical isolation between the first and second lines and being at least partially exposed such that, during use, a user's breath contacts exposed parts of the first lines simultaneous to exposed parts of the second lines. Voltage circuitry is configured to apply, during use, a voltage difference between the first and second lines. A comparator or controller is configured to detect when a current flows in the first and second lines due to a short circuit through pathogens from the user's breath bridging the electrical isolation between an individual first line and an opposing, individual second line. A warning module is configured to indicate when the comparator or controller detects the current flow above a warning threshold.

Additional features may be implemented in Apparatus B. By way of example, the exposed parts of the first and second lines may form a sensitive area of the sensor. Within the sensitive area, the first lines may be separated from the second lines by a distance of 50 nanometers or less.

The sensor may include third conductive lines alternated with fourth conductive lines, the third and fourth lines being formed over an isolation layer providing electrical isolation between the third and fourth lines and being at least partially exposed such that, during use, a user's breath contacts exposed parts of the third lines simultaneous to exposed parts of the fourth lines. The third and fourth lines may be formed at elevational levels over the first and second lines. The isolation layer may provide electrical isolation between the third/fourth lines and the first/second lines.

The first and second lines may be parallel to each other, the third and fourth lines may be parallel to each other, and the first and second lines may be orthogonal to the third and fourth lines.

Holes may be formed through the isolation layer between the third and fourth lines and provide the exposed parts of the first and second lines.

Holes may be formed through the isolation layer between the third and fourth lines, holes may be formed through the substrate between the first and second lines, and the isolation layer holes may be aligned with the substrate holes, providing a pathogen filter with the sensor.

A first, continuous conductive material may form all of the first lines, a second, continuous conductive material may form all of the second lines, and the first and second lines may be formed at a same elevational level over the substrate.

The sensor may include third conductive lines alternated with fourth conductive lines, the third and fourth lines being formed over an isolation layer providing electrical isolation between the third and fourth lines and being at least partially exposed such that, during use, a user's breath contacts exposed parts of the third lines simultaneous to exposed parts of the fourth lines. A third, continuous conductive material may form all of the third lines, a fourth, continuous conductive material may form all of the fourth lines, and the third and fourth lines may be formed at a same elevational level over the isolation layer. The elevational level of the third and fourth lines may be over the elevational level of the first and second lines and the isolation layer may provide electrical isolation between the third/fourth lines and the first/second lines.

Apparatus B may further include a conduit having an inlet configured to receive the user's breath and a channel from the inlet directed toward the sensor.

Apparatus B may further include at least one electrical power source electrically connected to the voltage circuitry.

Apparatus B may further include a filter configured to block particles having a size of 500 nanometers or greater from reaching the sensor.

The described additional features of Apparatus B may also be implemented in other devices and methods herein.

Although minima and/or maxima are listed for the above described ranges and other ranges designated herein, it should be understood that more narrow included ranges may also be desirable and may be distinguishable from prior art. Also, operating principles discussed herein may provide an additional basis for the lesser included ranges.

In compliance with the statute, the embodiments have been described in language more or less specific as to structural and methodical features. It is to be understood, however, that the embodiments are not limited to the specific features shown and described. The embodiments are, therefore, claimed in any of their forms or modifications within the proper scope of the appended claims appropriately interpreted in accordance with the doctrine of equivalents.

### TABLE OF REFERENCE NUMERALS FOR FIGURES

10 sensor
12 substrate
14 voltage line
16 voltage line
18 isolation
20 sensor
22 substrate
24 voltage line
26 voltage line
28 isolation
30 hole
32 isolation layer
34 voltage line
36 voltage line
38 isolation
40 sensor
42 hole
44 hole
46 hole
50 apparatus
52 housing
54 interior cone
56 inlet
58 display
60 sensor
62 electronic board
64 outlet
66 air flow
70 apparatus
72 sensor
74 amplifier
76 comparator
78 warning module
79 battery
80 apparatus
82 sensor
84 amplifier
86 converter
88 controller
90 display
92 warning module
94 battery

What is claimed is:

1. A pathogen detection method comprising:
providing a sensor including first conductive lines alternated with second conductive lines, the first and second lines being formed over a substrate providing electrical isolation between the first and second lines and being at least partially exposed;
via voltage circuitry, applying a voltage difference between the first and second lines;
applying a user's breath to the sensor and contacting exposed parts of the first lines simultaneous to exposed parts of the second lines;
pathogens from the user's breath bridging the electrical isolation between an individual first line and an opposing, individual second line and causing a short circuit;
via a comparator or controller, detecting a current flowing in the first and second lines due to the short circuit through the pathogens; and
via a warning module, indicating that the comparator or controller detected the current is above a warning threshold.

2. The method of claim 1, wherein the exposed parts of the first and second lines form a sensitive area of the sensor and, within the sensitive area, the first lines are separated from the second lines by a distance of 50 nanometers or less.

3. The method of claim 1, wherein:
the sensor comprises third conductive lines alternated with fourth conductive lines, the third and fourth lines being formed over an isolation layer providing electrical isolation between the third and fourth lines and being at least partially exposed;
the third and fourth lines are formed at elevational levels over the first and second lines and the isolation layer provides electrical isolation between the third/fourth lines and the first/second lines;
the voltage circuitry also applies the voltage difference between the third and fourth lines;
the user's breath to the sensor also contacts exposed parts of the third lines simultaneous to exposed parts of the fourth lines;
the pathogens from the user's breath also bridge the electrical isolation between an individual third line and an opposing, individual fourth line and cause another short circuit; and
the comparator or controller also detects a current flowing in the third and fourth lines due to the other short circuit through the pathogens.

4. The method of claim 3, wherein holes are formed through the isolation layer between the third and fourth lines and provide the exposed parts of the first and second lines.

5. The method of claim 3, wherein holes are formed through the isolation layer between the third and fourth lines, holes are formed through the substrate between the first and second lines, and the isolation layer holes are aligned with the substrate holes and the method comprises applying the user's breath through the aligned holes with the sensor acting as a pathogen filter.

6. The method of claim 1, further comprising receiving the user's breath at an inlet of a conduit and directing the user's breath through a channel of the conduit to the sensor.

7. The method of claim 1, further comprising electrically connecting at least one electrical power source to the voltage circuitry.

8. The method of claim 1, further comprising, via a filter, blocking particles having a size of 500 nanometers or greater from reaching the sensor.

9. A pathogen detection apparatus comprising:
a sensor including first conductive lines alternated with second conductive lines, the first and second lines being formed over a substrate providing electrical isolation between the first and second lines and being at least partially exposed such that, during use, a user's breath contacts exposed parts of the first lines simultaneous to exposed parts of the second lines;
voltage circuitry configured to apply, during use, a voltage difference between the first and second lines;
a comparator or controller configured to detect when a current flows in the first and second lines due to a short circuit through pathogens from the user's breath bridging the electrical isolation between an individual first line and an opposing, individual second line; and
a warning module configured to indicate when the comparator or controller detects the current flow above a warning threshold.

10. The apparatus of claim 9, wherein the exposed parts of the first and second lines form a sensitive area of the sensor and, within the sensitive area, the first lines are separated from the second lines by a distance of 50 nanometers or less.

11. The apparatus of claim 9, wherein:
the sensor comprises third conductive lines alternated with fourth conductive lines, the third and fourth lines being formed over an isolation layer providing electrical isolation between the third and fourth lines and being at least partially exposed such that, during use, a user's breath contacts exposed parts of the third lines simultaneous to exposed parts of the fourth lines; and
the third and fourth lines are formed at elevational levels over the first and second lines and the isolation layer provides electrical isolation between the third/fourth lines and the first/second lines.

12. The apparatus of claim 11, wherein:
the first and second lines are parallel to each other, the third and fourth lines are parallel to each other, and the first and second lines are orthogonal to the third and fourth lines.

13. The apparatus of claim 11, wherein holes are formed through the isolation layer between the third and fourth lines and provide the exposed parts of the first and second lines.

14. The apparatus of claim 11, wherein holes are formed through the isolation layer between the third and fourth lines, holes are formed through the substrate between the first and second lines, and the isolation layer holes are aligned with the substrate holes, providing a pathogen filter with the sensor.

15. The apparatus of claim 9, wherein a first, continuous conductive material forms all of the first lines, a second, continuous conductive material forms all of the second lines, and the first and second lines are formed at a same elevational level over the substrate.

16. The apparatus of claim 15, wherein:
the sensor comprises third conductive lines alternated with fourth conductive lines, the third and fourth lines being formed over an isolation layer providing electrical isolation between the third and fourth lines and being at least partially exposed such that, during use, a user's breath contacts exposed parts of the third lines simultaneous to exposed parts of the fourth lines;
a third, continuous conductive material forms all of the third lines, a fourth, continuous conductive material forms all of the fourth lines, and the third and fourth lines are formed at a same elevational level over the isolation layer; and
the elevational level of the third and fourth lines is over the elevational level of the first and second lines and the isolation layer provides electrical isolation between the third/fourth lines and the first/second lines.

17. The apparatus of claim 9, further comprising a conduit including an inlet configured to receive the user's breath and a channel from the inlet directed toward the sensor.

18. The apparatus of claim 9, further comprising at least one electrical power source electrically connected to the voltage circuitry.

19. The apparatus of claim 9, further comprising a filter configured to block particles having a size of 500 nanometers or greater from reaching the sensor.
